(19) United States
(12) Patent Application Publication
(10) Pub. No.: US 2019/0083299 A1
(43) Pub. Date: Mar. 21, 2019

(54) WEARABLE THERAPEUTIC APPARATUS

(71) Applicant: Kathyrn Phyllis Rozanski, Auburn, MA (US)

(72) Inventor: Kathyrn Phyllis Rozanski, Auburn, MA (US)

(21) Appl. No.: 15/884,591

(22) Filed: Jan. 31, 2018

Related U.S. Application Data
(60) Provisional application No. 62/559,808, filed on Sep. 18, 2017.

(51) Int. Cl. A61F 7/00; A61F 9/04; A61F 7/02

(52) U.S. Cl. CPC A61F 7/0053; A61F 9/04; A61F 2007/0228; A61F 2007/0054; A61F 2007/0004; A61F 7/02

(57) ABSTRACT

A therapeutic eyewear apparatus is provided for selectively providing continuous long-term temperature-based therapy to the eye region of a wearer. The eyewear apparatus provides an eye mask adapted to be worn over the eye region of the wearer, wherein the eye mask houses a fluidly-controlled temperature element operatively associated with a control unit. The control unit provides a fluid reservoir, pump, temperature and circulation controls for selectively circulating temperature-controlled fluid through the temperature element. The temperature element may be a conduit fluidly coupled to the control unit, wherein the conduit tortuously travels through a cover of the eye mask.

### CROSS-REFERENCE TO RELATED APPLICATION

This application claims the benefit of priority of U.S. provisional application No. 62/559,808, filed 18 Sep. 2017, the contents of which are herein incorporated by reference.

### BACKGROUND OF THE INVENTION

The present invention relates to wearable therapeutic devices and, more particularly, to a therapeutic eyewear apparatus selectively providing continuous long-term temperature-based therapy to the eye region of a wearer.

In cases where a person needs continuous ice applications over the eyes to prevent swelling (such as after surgery) the eye masks that are available today require constant attention in order to be effective. Furthermore, there is nothing on the market that provides continuous warm/hot or cold/cool eye compress treatments for persons who may have eye surgery or who suffer from dry eye or other eye conditions requiring heat or cold therapy. Other eye masks provide only limited time spans before they either cool off (when heat therapy is ordered) or warm up (when cold therapy is ordered).

As stated, other eye masks provide only a short time frame of heat or cold therapy. A person may need more sustained, continuous heat or cold applications in order to benefit from the therapy, so as to heal faster and/or avoid swelling.

As can be seen, there is a need for a therapeutic eyewear apparatus selectively providing long term hot and cold therapy for continuous treatment required for decreasing swelling (cool/cold treatment) or increasing mybomien gland function (warm/hot treatment). The present invention can be applied by the patient or a caregiver to facilitate the healing process.

### SUMMARY OF THE INVENTION

In one aspect of the present invention, a therapeutic eyewear apparatus, includes an eye mask providing a surface adapted to interface with an eye region of a human wearer of the eye mask; and a conduit operatively associated with said surface, wherein a temperature of the conduit is fluidly controlled.

In another aspect of the present invention, the therapeutic eyewear apparatus includes an eye mask providing a surface adapted to interface with an eye region of a human wearer of the eye mask; a conduit operatively associated with said surface, wherein a temperature of the conduit is fluidly controlled; a control unit fluidly coupled to the conduit, wherein the control unit provides a fluid reservoir and pump fluidly coupled to the conduit for selectively providing temperature-controlled fluid; and a removable connection for interfacing the eye mask to the eye region so as to provide selective compression between the surface and the eye region.

These and other features, aspects and advantages of the present invention will become better understood with reference to the following drawings, description and claims.

### BRIEF DESCRIPTION OF THE DRAWINGS

FIG. 1 is a front elevation view of an exemplary embodiment of the present invention in use;

FIG. 2 is a top plan view of an exemplary embodiment of the present invention in use;

FIG. 3 is a detail top plan view of an exemplary embodiment of the present invention, as indicated by the line 3-3 of FIG. 2;

FIG. 4 is a cross-sectional view of an exemplary embodiment of the present invention, taken along line 4-4 of FIG. 1; and

FIG. 5 is a front elevation view of an exemplary embodiment of the present invention.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a therapeutic eyewear apparatus selectively providing continuous long-term temperature-based therapy to the eye region of a wearer. The eyewear apparatus provides an eye mask adapted to be worn over the eye region of the wearer, wherein the eye mask houses a fluidly-controlled temperature element operatively associated with a control unit. The control unit provides a fluid reservoir, pump, temperature and circulation controls for selectively circulating temperature-controlled fluid through the temperature element. The temperature element may be a conduit fluidly coupled to the control unit, wherein the conduit tortuously travels through a cover of the eye mask.

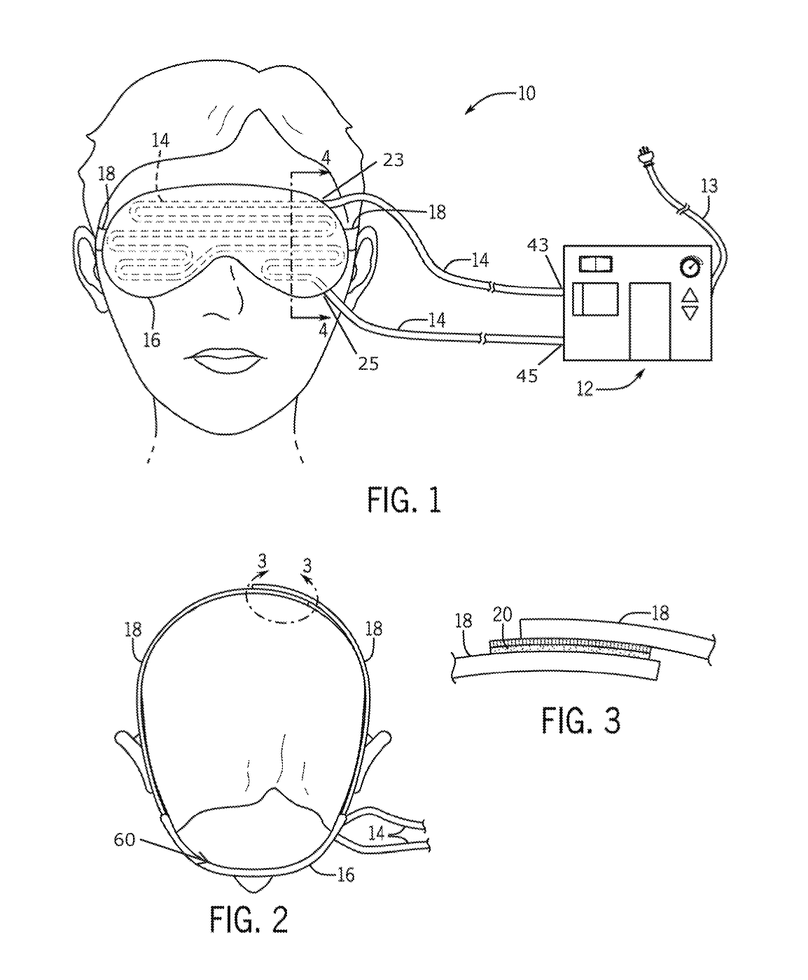

Referring to FIGS. 1 through 5, the present invention may include a therapeutic eyewear apparatus 10 housing a conduit 14 operatively associated with a control unit 12 adapted to selectively provide an array of temperatures over a sustained and continuous duration.

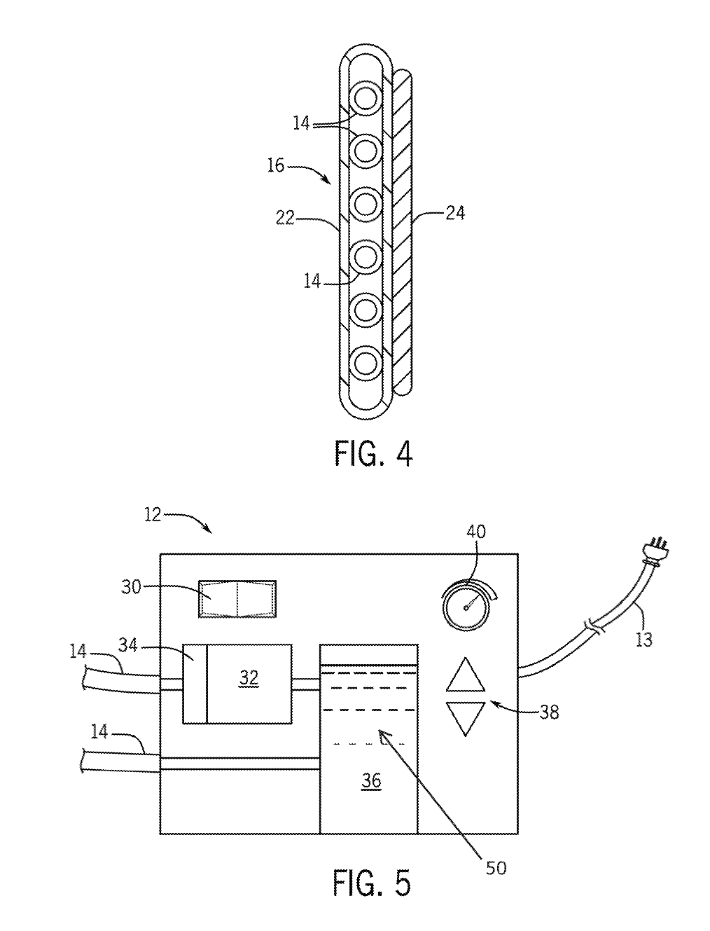

The therapeutic eyewear apparatus 10 includes an eye mask 16 dimensioned and adapted to be located over the eyes of a human wearer, as illustrated in FIG. 1. The eye mask 16 may provide a cover 22 for housing the conduit 14. The cover 22 may be made of comfortable material, such as cloth. The eye mask 16 may also provide a pad 24 disposed on a surface that interfaces with the wearer, as illustrated in FIG. 4.

Referring to FIG. 1, the conduit 14 may enter the cover 22 through an inlet 23 and exit through an outlet 25, wherein the inlet 23 is fluidly coupled to an output 43 of the control unit 12, and the outlet 25 is fluidly coupled to an input of the same control unit 12. The conduit 14 may make a tortuous path through the cover 22 so as to provide substantial coverage over both eyes, the eyebrow portions and the portion between the eyes of a wearer during use, i.e., eye region, as illustrated in FIG. 1.

Referring to FIG. 5, the control unit 12 provides a fluid reservoir 36 fluidly coupled to the input 45 and output 43. A pump 32 and filter 34 may be disposed between the fluid reservoir 36 and the output 43. The control unit 12 may provide a temperature control 38 and a circulation control 40 operatively associated with the fluid reservoir 36 and the pump 32, respectively. The control unit 12 may have an internal power source (e.g., batteries) or be electrically connected 13 to an external power source, the control unit 12 providing a power on-off switch 30 to control the power source.

Referring to FIGS. 2 and 3, the eye mask 16 may provide connectors 18 with removable fasteners 20 for removably attaching the eye mask 16 to the wearer, whereby the eye mask 16 would be adjustable to various sized heads. The connectors 18 may be straps or the like for operatively engaging the eye mask 16 to the wearer, whereby a user may selectively provide a compressive force at the interface surface 60 of the eye mask 16 and wearer.

A method of using the present invention may include the following. The therapeutic eyewear apparatus 10 disclosed above may be provided. A user could be anyone requiring heat or cold therapy to the eyes. Oftentimes, heat therapy is prescribed for someone with dry eye (helps to keep open mybomian glands) or someone may need cold compresses following eye surgery (helps reduce the amount of swelling). The idea is to take an eye mask 16 controllable by the control unit 12 adapted to circulate either hot/warm or cool/cold fluid 50 (saline or sterile water) through the conduit 14 embedded in the eye mask 16. The temperature control 38 enables a user to regulate the amount of heat or cold that circulates. The eye mask 16 and conduit 14 would be non-latex material. The circulated fluid 50 would circulate continuously through the conduit 14/channels within the eye mask 16, thereby selectively maintaining a determined temperature.

The user would have the benefit of having continuous heat or cold therapy. The user would not have to stop to reheat or cool the eye mask 16, depending on which type of therapy the person requires. A user would be able to use this independently and would therefore not need assistance from anyone, which would be beneficial to the user who may be recovering from surgery or other procedures. They would not need to get up to reheat or add a cold source to the eye mask 16 as it would be controlled by the fluid 50 circulating through the mask 16 through the control unit 12, which could be placed at the bedside.

Additionally, the selectively determined temperature and circulation could be control by a computer device (not shown) and associated program product. The present invention could take the form of headwear or even bodywear to selectively provided temperature-based therapy to many other parts of a wearer.

It should be understood, of course, that the foregoing relates to exemplary embodiments of the invention and that modifications may be made without departing from the spirit and scope of the invention as set forth in the following claims.

What is claimed is:

1. A therapeutic eyewear apparatus, comprising:
an eye mask providing a surface adapted to interface with an eye region of a human wearer of the eye mask; and
a conduit operatively associated with said surface, wherein a temperature of the conduit is fluidly controlled.

2. The therapeutic eyewear apparatus of claim 1, further comprising a control unit fluidly coupled to the conduit for selectively providing temperature-controlled fluid.

3. The therapeutic eyewear apparatus of claim 2, wherein the control unit provides a fluid reservoir and pump fluidly coupled to the conduit.

4. The therapeutic eyewear apparatus of claim 1, further comprising a removable connection for interfacing the eye mask to the eye region.

5. The therapeutic eyewear apparatus of claim 4, wherein the removable connection is adapted to provide selective compression between the surface and the eye region.

6. The therapeutic eyewear apparatus of claim 1, further comprising a pad along the surface for interfacing with the eye region.

7. The therapeutic eyewear apparatus of claim 1, further comprising a cover for housing the conduit.

8. A therapeutic eyewear system, comprising:
an eye mask providing a surface adapted to interface with an eye region of a human wearer of the eye mask;
a conduit operatively associated with said surface, wherein a temperature of the conduit is fluidly controlled;
a control unit fluidly coupled to the conduit, wherein the control unit provides a fluid reservoir and pump fluidly coupled to the conduit for selectively providing temperature-controlled fluid; and
a removable connection for interfacing the eye mask to the eye region so as to provide selective compression between the surface and the eye region.

9. The therapeutic eyewear apparatus of claim 8, further comprising a pad along the surface for interfacing with the eye region.

10. The therapeutic eyewear apparatus of claim 8, further comprising a cover for housing the conduit.
